SEQUENCE LISTING (9 sequences)

SEQ ID NO: 1 - DNA, 15999 bp, Artificial Sequence
  The sequence is synthetized.
  misc_feature (1)..(8): Not1 restriction site (synthetic sequence)
  misc_feature (9)..(370): Homologous arm (human sequence)
  misc_feature (433)..(1444): Mouse IgH 5′ enhancer (mouse sequence).
  misc_feature (3278)..(8527): mouse Iggamma1 conversion element (Sgamma1) (mouse sequence).
  misc_feature (8648)..(8692): Human Iggamma1 Hinge (human sequence)
  misc_feature (8811)..(9141): Human Ig  gamma1 CH2 (human sequence).
  misc_feature (9238)..(9560): Human Ig gamma1 CH3 (human sequence).
  misc_feature (10862)..(10992): Mouse TM1 (mouse sequence).
  misc_feature (11810)..(11890): Mouse TM2 (mouse sequence).
  misc_feature (12611)..(13477): Mouse PolyA signal sequence (mouse sequence).
  misc_feature (13837)..(13870): Lox (synthetic sequence).
  misc_feature (13871)..(15192): pGK-Puromycin screening genes (synthetic sequences).
  misc_feature (15193)..(15226): Lox (synthetic sequence).
  misc_feature (15227)..(15508): Homologous arm (homologous recombination for linking to mouse 3′-LCR) (mouse sequence).
  misc_feature (15509)..(15516): AsiSI (synthetic sequence).
  misc_feature (15517)..(15991): Homologous arm (homologous recombination for linking to human Ig C-region sequence) (human sequence).
  misc_feature (15992)..(15999): Not1 (synthetic sequence)
gcggccgccgagatgcctgaacaaaccaggggtcttagtgatggctgaggaatgtgtctcaggagcggtgtctgtaggactgcaagatcgctgcacagcagcgaatcgtgaaatattttctttagaattatgaggtgcgctgtgtgtcaacctgcatcttaaattctttattggctggaaagagaactgtcggagtgggtgaatccagccaggagggacgcgtagccccggtcttgatgagagcagggttgggggcaggggtagcccagaaacggtggctgccgtcctgacaggggcttagggaggctccaggacctcagtgccttgaagctggtttccaagagaaaaggattgtttatcttaggaggcatcttagtgattgagtcaagggagaaaggcatctagcctcggtctcaaaagggtagttgctgtctagagaggtctggtggagcctgcaaaagtccagctttcaaaggaacacagaagtatgtgtatggaatattagaagatgttgcttttactcttaagttggttcctaggaaaaatagttaaatactgtgactttaaaatgtgagagggttttcaagtactcatttttttaaatgtccaaaattcttgtcaatcagtttgaggtcttgtttgtgtagaactgatattacttaaagtttaaccgaggaatgggagtgaggctctctcataacctattcagaactgacttttaacaataataaattaagtttcaaatatttttaaatgaattgagcaatgttgagttggagtcaagatggccgatcagaaccagaacacctgcagcagctggcaggaagcaggtcatgtggcaaggctatttggggaagggaaaataaaaccactaggtaaacttgtagctgtggtttgaagaagtggttttgaaacactctgtccagccccaccaaaccgaaagtccaggctgagcaaaacaccacctgggtaatttgcatttctaaaataagttgaggattcagccgaaactggagaggtcctcttttaacttattgagttcaaccttttaattttagcttgagtagttctagtttccccaaacttaagtttatcgacttctaaaatgtatttagaattcattttcaaaattaggttatgtaagaaattgaaggactttagtgtctttaatttctaatatatttagaaaacttcttaaaattactctattattcttccctctgattattggtctccattcaattcttttccaatacccgaagcatttacagtgactttgttcatgatcttttttagttgtttgttttgccttactattaagactttgacattctggtcaaaacggcttcacaaatctttttcaagaccactttctgagtattcattttaggagaaagactttttttttaaatgaatgcaattatctagacttatttcagttgaacatgctggttggtggttgagaggacactcagtcagtcagtgacgtgaagggcttctaagccagtccacatgctctgtgtgaactccctctggccctgcttattgttgaatgggccaaaggtctgagaccaggctgctgctgggtaggcctggactttgggtctcccacccagacctgggaatgtatggttgtggcttctgccacccatccacctggctgctcatggaccagccagcctcggtggctttgaaggaacaattccacacaaagactctggacctctccgaaaccaggcaccgcaaatggtaagccagaggcagccacagctgtggctgctgctcttaaagcttgtaaactgtttctgcttaagagggactgagtcttcagtcattgctttagggggagaaagagacatttgtgtgtcttttgagtaccgttgtctgggtcactcacatttaactttccttgaaaaactagtaaaagaaaaatgttgcctgttaaccaataatcatagagctcatggtactttgaggaaatcttagaaagcgtgtatagaaagtagggactctggggtcccaggttcaatcccagcattgttattacagtaactaagggaacagaagcaactctggattgaaggagggaaagacaagtagagggaagcccaagctgctctgagggactcagggtatgtgggggatccagagagccaggggaactcgagagctcaggacagaagactgagcacctgcaaaagagctgaggctggtaagagtaacaaggtaacctgggtaacaggttcagcagctccaggggagccaggagagatggaagtgtggggagccaggcagagcagctccatgacagccaggacaggtggaagtgtggtacccaggcagagcagctccatgacagccaggacaggtggaattgtggtgacccaggcagagcagctccagggcagccaggacaggtggaagtgtggtgacccaggcagagcatctataggggagccaggacaggtgggagtgtggtgacccaggcagagcagctccagggcatccaggacaggtggaattgtggtgacccaggcagagcagctacaggggagccaggaaaggtgggagtgtggtgacccaggcagagcagctacaggggagccaggacaggtgggagtgtggtgacccaggcagagcagctatagggcagccaggacaggtggaagtgtggtgacccaggcagagcagctacaggggagccaggaaaggtgggagtgtggtgacccaggcagagcagctacaggggagccaggaaaggtgggagtgtggtgacccaggcagagcagctacaggggagccaggaaaggtgggagtgtggtgacccaggcagagcagctacagggcagccaggacaggtggaagtgtggtgacccaggcagagcagctccatggcacccaggacaggtggaagtgtggtgacccaggcagagtagctacagggcagccaggacaggtggaagtgtggtgacccaggcagagcagcttcagggcagccaggacaggtggaagtgtggtgacccaggcagagcagcttcagggcagccaggacaggtggaagtgtggtgacccaggcagagcagcttcagggcagccaggacaggtgggagtgtggtgacccaggcagagcagctccaggggagccaggacaggtgggagtgtggtgacccaggcagagcagctccagggcagccaggacaggtggaagtgttgtgacccaggcagagcatctatagggtagccaggacaggtggaattgtggtgacccaggcagagcagctccagggcagccaggacaggtgggagtgtggtgacccaggcagagcatctataggggagccaggacaggtggaagtgtggtgacccaggcagagtagctacagggagccaggacaggtggaagtgtggtgacccaggcagagtagctcttggggagcctggacaggtgggagtgtggtgacccaggcagagcagctacaggggagctaggacaggtggaagtgtggtgacccaggcagagtagctcttggggagccaggacaggtggaagtgtggtgacccaggcagagcatctataggggagccaggacaggtgggagtgtggggatccaggtaaggcaggactggggagccaggacaggtggaagtgtggtgacccaggcagagcagctccagggcagccaggacaggtggaagtgtggtgacccaggcagagcagctccatggcagccaggacaggtggaaatgtggtgacccaggcagagcagctccagggcagccaggacaggtggaagtgtggtgacccaggcagagcatctataggggagccaggacaggtgggagtgtggtgacccaggcagagcagctccatggcagccaggacaggtgggagtgtggtgacccaggcagagcagctcatggcagccaggacaggtgggagtgtggtgacccagtcagagcagctgcagggcagccaggacaggtgggagtgtggtgacccaggcagagcatctataggggaaccaggacaggtggaagtgtggtgacccaggcagagcagctgcagggcagccaggacaggtgggagtgtggtgacccaggcagagcatctataggggaaccaggacaggtggaagtgtggtgacccaggcagagcagctatagggagccaggacaggtggaagtgtgttgatccagacaaaacacctacaggggagacaaatcagctagattgggcggatcctggcagatcagcttcaggggacctaggcaagtggaactgtggggacccgtgtagggcagctgtagggaaatcaggacaggtacaagtgtgtggatccatgcagtgtagtgccttgggagcctgaacagatagaagggtggggatacaggcagggtagctatagggaatccagttgaggtggaagaatggggatccaggcagagtagctatagggcagccaggagaaatggaagaatgcagatccaaacagaagagctacagaggagccaagacaactagaagtgtgtgaatccaggcagagcagtgccttaggagcaaggacagggaagctatagggaaaccaggacaggtggaagaatggggatccaggtgctgcagctacaggtaagcagggacaggtggaagtgtggagacccaggcagagcagctatagggcagccaggacaggtgggagtgtggggatccaggtaaggcaggactggggagtcaaggtaggtggaatgtgaagttccaggcagaacaggtccagggtgccaggacaggtacaagtttagtagttatagaggaacaggggcaggttagaatgaaggatgggcatcccgggtgagcaaatacaagggaactgatggcaaatggaagggcagggacccagactaaatggctacagagaagctgaggcaggtaagagtgtgggaacccagtcaaaaaccacagaagagcaggagctaattggcacgggctggggtgcatgctggctactcatagggaagctgggataagtagtagttggggattctaagcagtcacagagaagctgatcccggtgagagtacggggtacacagctgagcaaatactacatagctggagctgatggctgtataaggtaccaggctgagcagctgaaggtaacctggacctagtgggggtgtgggagacctggctgagcagctaccaaggatcagggatagacatgtaagcagtcaagctcagctactacatgagagctggagctagtatgaaggtggaggtccagttgagtgtctttagagaaactgaggcaagtgggagtgcagagatccaggctgagcagctccagcttagctggtataggtgacaggatgggggataacaaggctaagaacacacagagagcaggatctcctgggtaggttacaggtcaagactgagtagaatcaggggagctgaggttgggagtaatgcagaattccagacttagcagtccaggcaaactaaaccaagtgggagtgtgggagtcctaactgaacaaataccaggcatatgaagctgataagtgtgtataaagtaccaagctgagcagctacaggagagctgggatagctatgtggggagaccaggttaagcaaacagtggagagcaagataaagtcttaatgtatgcatccaggctgaatagacacaggggagctgaggaaggcagtactagaggattctaggcttagaagtcacagggaaactgaggcctgggtgagggtgggcatcctagctggaaaaatcaccagggagctggagctgatgggtataaaaaggtaccaggttgagcagctacaggagagctaggacatgtggggatgttttgttccaggctgaacaactgtagagcatcagggggaggtggaactttaagaagtcaggctgagcagctacaggagagctgcagctattcggtatgtggaggtccagccagagcagttacagggtagctgggataaatggggctggagaaccaggctaagaagacacaggggagcaggttctagtctgcataggagtggggatccaggtcgagtacacacagaagagaaaagggtaggtagtaatggaggattctagactcagctataactgagtggtagtgtgagtgttctagctggaaaaaaaaataccatgaagctggagctgatgggtataaaaggaacgagcctgagaaactgtagggtatctggggtggatggggatgtggggagtcatgctgagctgctacaagggcagtgtggcctctaggagtgtaggggaccaaactgagcatctacagggaaactgaggcaaactggagtgtagggctccagaatgagcaactgcaggtcagctgaggccggtaagagtgtggggaaccagactggacagttagttgcaagttagccagggtaggtgagagttctattagagaagcctcagcagaatgggaagtgggaactaggaaaccaggatgagcacctaacaacagaagtgaggcaggttagggtgtaggggatgagctgtgcagctactgtggagcacgcagactggaatagaagaggttctggctgaacacttgaagggaaccaggtaggcaggaggcagtgtgtacagacagctgaatgagacatcatgcagggccagttccctgccctgagctacattagctgggaccagggccagcggttgaggaaccaggcagaggtaaaatggtggtgtgataggaagccaatggcagaggggagggagaagttatgcttatgtcatgctggaatgtaggaaggggaaagagccaggatgtctaggctggagctgatccggctgtctgctctgatggcagcaacaggcctgagcttctctggactcaagaagccagggcaacaaaataaagggggcctagcagagcaaagacactgctagcactgggatcaggaaaacaggacaagactcccgatccaggaggtcatgggagggaaggagaagactacaggggactgtccttgggaaagagtaagggcccactggagggagtgctcaggaagcaagcccattgacaggggagaacaaggctggtggacgtctggatgggcagtaggcagccccaagtcccaggagggagagaagaggcagataggaaaacaggtcaggtctagcagaggcctactgaagtactctcctcaggacagaaccctgaatactggaaaatgcggaactgctgcaggcacaaagaatagctgaggtctaagagtaaaacagactaggggatgagaggaccttaggaagagcctttggctgagcaggaacaagaacaggggaaatcctagggctgacattgccagtggaaacatacaggctggagctctttagtcaggagctccagctgtgatctagacatcaggcaggaagatcaaatctgtcccaacaatacaggggacagaggctcaacctagagtgtgagcatcaggggctgtgcaggagatttcagagctcaggtgcagcagagactagcatggccctggggataaagggaaggatccaagggacaaggggataatcctggggaggtaagggccagcttcgtgacagaaggtggtggtgtccaacttcaagagccctgtgctacaatttaaaaaaaaaaaaaaaggaaagggacttctctgtgtttggcaacacaagtgcgatgcacaggcaggaagatcaaatctgtcccaacaatacaggggacagagggtcaacctacaaaaggaaagaacctggggcagtgtgaagacaacactgtagaagccaaggctgagttcactgagctctcgttagtgagactacacagcaaggaggtggcgggcactgagcagtgaggccccgggaagtgggggtgatggtggtgacggtggtaactgttaagaactgggggaaagaattgtggagaaccaagctaaatagttatgtcaaaccacatgtttaggagcctgggttgacttcatagggagtaggcatggaggctaatctagaggtttgtgtataggcaagaagtgaatcctgacccaagaatagagagtgctaaacggacttagttcaaagacaactgaaaaagacaatgcctgcaaaacaaagctaaggccagagctcttggactatgaagagttcagggaacctaagaacagggaccatctgtgtacaggccaaggccggtagaagcagcctaggaagtgtcaagagccaacgtggctgggtgggcaaagacaggaagggactgttaggctgcagggatgtgccgacttcaatgtgcttcagtattgtccagattgtgtgcagccatatggcccaggtataagaggtttaacagtggaacacagatgcccacatcagacagctggggggcgggggtgaacacagatacccatactggaaagcaggtggggcattttcctaggaacgggactgggctcaatggcctcaggtctcatctggtctggtgatcctgacattgataggcccaaatgttggatatcacctactccatgtagagagtcggggacatgggaagggtgcaaaagagcggccttctagaaggtttggtcctgtcctgtcctgtctgacagtgtaatcacatatactttttcttgtagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaatgataatgtcctggtgatttctgagatgtagagtctagctaggtcatggaatgaggggtctccatggtttgaggcctgagttgtgactaaggaaaaacccataggcctacactgccacacccagcacttttgaatttgcctgacatgaaaagaatttacctctccctggaaagtggagccttatccctaggcagttcccttaccagaccttcctctagcttgcactttgttctgggcacagaatgtgtctaaccccccaaagcaaggaagacacaacctctacctccctcactctgtccttaccccttttcctggctaagcatctcactgagtgcgctgaatagatgcatgtggccacagtcttgcagacagacccttgccatctctccactcagctttccagaggctaagtctagcccgtatggtgataatgcagggagctctatgctatctcagtgctatcagactcccaagtggaggatgaacatggacccattaaaaccaacctgcgcagcaacaccctgccaataaggcccgtatgtgaaaatgtgcacacatctacacatgcacaggcacacacacacacacatgcatgggcacacacacatacagagagagagaatcacagaaactcccatgagcatcctatacagtactcaaagataaaaaggtaccaggtctacccacatgatcatcctcggcatttacaagtgggccaactgatacagataaaacttttctatgccaaggacgccaacatatacacaagtccgctcatgacaaatctgtccctgaacctcagactggcgcccgtgactcatacagtggacactcctccaaagctgtatagcttcctttacttccctgtgtgtactttctctgaagtacactcatcacacagaagaggccctgtgattactctggccctctgttcttggtcatcagagaatagacagaagatcaggcaaactacacagacacttcccacaatcatcacaggccctgactctgctctccagtctcaaaactgaaggctggagcacacagaataagctcctgcacaggccaggccagtatcgggtccagtgtgtctgactgagcccagggacaaaatggcagcactttggggaactgaggtttctggtccaagaaggagagatggaggcccagggagggtctgctgacccagcccagcccagcccagctgcagctttctcctgggcctccatacagcctcctgccacacagggaatggccctagccccaccttattgggacaaacactgaccgccctctctgtccagggctgcaactggacgagacctgtgctgaggcccaggacggggagctggacgggctctggacgaccatcaccatcttcatcagcctcttcctgctcagtgtgtgctacagcgctgctgtcacactcttcaaggtcagccatactgtccccacagtgtctacaatgtcctcatactcttccccatactgtccctgtggtgacctataccccacactgtcccatgctaatgaccacagtcttacatgctatgtaatgctgtctacccttctgtatgcacagtctcacaatgtcccatgcagtctccacgatgctccatactgtccccattccaacccatgctgccccttgttccccgctatgctgtcccatgctattgtctgtattttcatgctcttttcacactgtccctagtgtcacattctgcccatgttgtccaccacattgtccccactctgtacacagcctcacactgtaccctgctacccgataatgttccctgttgtccccaactctctccctgcaccatttgtcaactgtcccctgaattcccatgttgttcccacactgttagtgtgtaatgtgctctgtcccaggtgtaccttgttccgtgctgtctcacttcatcgcccattctgtccttgtactaaccccactctatcaccacactgtccctatgcactgcccacattgtcctcatactgtcccattttgtatcttcatcctgtccccatagtgtccaatgatctaccccacactattcccacttcatgcccctacaatttccctattccattcctctctggtcaccatgccatccttcccactcctgcacagctggagagggactcccgggatgagtccttgcccagatgagctacctatctagaggagtcttcaggtgggaagggaatgcagtcttgatcttggtcttattcaccctgtctcacaggtaaagtggatcttctcctcggtggtggagctgaagcagacactggttcctgaatacaagaacatgattgggcaagcaccctaggccacctcctgtaatggcatttcccaggccccgaaggaccctgtccaatatgccaagcagcacaactgagatcacactgtctgctcatctcgctttcctccgaccccgagactcagctactctcaaattttccctctctgaaggaccatgtggacattacattgctccaggccacagccaccaggacctaaaacaccatcacagcagcaccaaagacactggatagacccacaagggcaatagtttcctcaacagtatatccaaactgttgggacaaacgagcaatcactgaagaagtgacaagttcccacaatgtcagtgtccagctgagaagggacaaaaagtggtaccagccctgtccacaccaccttctaattcacaggaatacgtgatagaagaggcaggttgtagatccgaaagatgagacagattttatcaactccagaaagagctgggtccaactgaattattctagcgaccttggcattgtcatgacctgccatgaccttcctccttaacacttcgataaaccctgggatatggaaaatgcctgtgtttctcagggtttgggaaagaaccatccatgttgggattcttgtgtagatcctccttctggtcacagatgcaatacactggattttcaggcaaaggagcaaattcacagacaactctggccctacagccctaagacctagacaccaccatctccttggaattatcaaatttaacacccggcacacaacaaagaaggactgggactttgaggcctttgtgtagccctagagggggcagaggccactgagcagggattgggtgatcacaaggacctcctggagagggacctgaggagcaggttccaattgggccaaagaaagaagaacaacaatagagatgaaggatgctggaaagagccatggtacagcagtcttgtccttcagacatgactcttacagcccaggactcttacagtagctagctggagcagaagtccaagggattaccatgccctagggccacaggctactggagggtggagtgagtctactacacaggtccaatgcctgtttctccattgcttctcagccaatgagaaatcagagtctccacctccaagaaaaaggaaggtggaaatgaaaggtgagcacctgccttcccgtgactggcagaaagatctccacggactcaaggctttgacttcaaacttcccgtagatccctgatgtctttaaggactctgtctgctttgttggttttgtttgtttgttttgtactttgtcaataaaacatttttcaaatattctacaattgctgtgctcttcctatgcaaactggccctggcacttcaaaacatggcacagttaagttgaccagtgggccatgcagagcatactacccctcctggtcctgtgtcctaccagatatcctctaagtgtcccttcactgctgggctccagttctgctgccctgaaccacacaggtcctcagtctgtcctccctatgggcagtttcatcccagccagaagctgccctgtggcccctaggctgcccaggcatggtctcccacaccaaccacacaaactaagaagcctgtcccatacattgacctcaggctcagtgataacatttctatagaaaaaggaaaggaataagaaaaaaaaaactccatattacatgggggctgcctaccttgctgatgcttattgtctcaagcaagtttggagaagttccaaatcaggcactgacagggtgggttctcagccatgctcttcttacctggtaggtgccttctcccccatggcacctcacaggctctccatctgtgtgtgtctgggtcctgatctcttctcataagtacaaagtcaggctggaagaggtacaccctagccctcattataacttaccagtttatgatcctgtctgcaaacatctgaggtccctgtggctataacttcgtataatgtatgctatacgaagttatctaccgggtaggggaggcgcttttcccaaggcagtctggagcatgcgctttagcagccccgctgggcacttggcgctacacaagtggcctctggctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcagcacgtgttgacaattaatcatcggcatagtatatcggcatagtataatacgacaaggtgaggaactaaaccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagcgggactctggggttcgaataaagaccgaccaagcgacgtctgagagctccctggcgaattcggtaccaataaaagagctttattttcatgatctgtgtgttggtttttgtgtgcggcgcgccgtttaaacgcggataacttcgtataatgtatgctatacgaagttatagaccgacgtgacaacttcccagcccactcacagactttcctgagcaataaattgatgcaaaaccacaaattcctactctcaaaaacaaacattaacaaaggattgggggagggggtcaggggtagtatggtggcgttgggcagggtaaactcagggtacccattgtctaatgtctgagacataacttgaacatatgtgtagctgcagccaaagatgaacaagtgatggtatttgtgtcctcttcagacccgataccaggtcattaagcttgagatctggacgcgatcgccagcctccagaatggtgggaaataagtttctgttgtttctcagccaccacgtctgtagtatgtggaagtcatcagaatcaaaattgagtcacctgtggttttttttttttctaaatccctgacaaatagagcctaggaaggccaagaagagaagagggttctcatccataaacacttgataacaaaaactatcaccaaggactctacaaaaactgcaactggcacaaagaccatcacaaccttacacagaaagtacttctgtgaggacatcttcccagcaacgggctgtccaacctcagactggcattgcctttgttattggtccttgtagagagggtaattatctcaaagcaatcatgtaatcctcctcatttttcctttgaaagccttggtctccctttgcctccctgaatacgcacatagctgatcatggcaggtgtatcccactgcagtgctctacctccaaatagatatcgcggccgc

SEQ ID NO: 2 - DNA, 11862 bp, Artificial Sequence
  The sequence is synthetized.
  misc_feature (1)..(8): Not1 restriction site (synthetic sequence).
  misc_feature (9)..(370): Homologous arm (human sequence).
  misc_feature (433)..(1444): Mouse IgH 5′ enhancer (mouse sequence).
  misc_feature (2550)..(4451): Mouse IgM conversion element (Smu) (mouse sequence).
  misc_feature (5674)..(6012): Mouse IgM CH2 (mouse sequence).
  misc_feature (6292)..(6609): Mouse IgM CH3 (mouse sequence).
  misc_feature (6717)..(7111): Mouse IgM CH4 (mouse sequence).
  misc_feature (7112)..(7341): Mouse PolyA signal sequence (mouse sequence).
  misc_feature (8910)..(9025): Mouse TM1 (mouse sequence).
  misc_feature (9144)..(9149): Mouse TM2 (mouse sequence).
  misc_feature (9265)..(9556): Mouse PolyA signal sequence (mouse sequence).
  misc_feature (9700)..(9733): Lox (synthetic sequence).
  misc_feature (9734)..(11055): pGK-Puromycin screening gene (synthetic sequence).
  misc_feature (11056)..(11089): Lox (synthetic sequence).
  misc_feature (11090)..((11371): Homologous arm (homologous recombination for linking to mouse Ig gamma1 Sgamma1) (mouse sequence).
  misc_feature (11372)..(11379): AsiSI (synthetic sequence).
  misc_feature (11380)..(11854): Homologous arm (homologous recombination for linking to human Ig C-region sequence) (human sequence).
  misc_feature (11855)..(11862): Not1 (synthetic sequence).
gcggccgccgagatgcctgaacaaaccaggggtcttagtgatggctgaggaatgtgtctcaggagcggtgtctgtaggactgcaagatcgctgcacagcagcgaatcgtgaaatattttctttagaattatgaggtgcgctgtgtgtcaacctgcatcttaaattctttattggctggaaagagaactgtcggagtgggtgaatccagccaggagggacgcgtagccccggtcttgatgagagcagggttgggggcaggggtagcccagaaacggtggctgccgtcctgacaggggcttagggaggctccaggacctcagtgccttgaagctggtttccaagagaaaaggattgtttatcttaggaggcatcttagtgattgagtcaagggagaaaggcatctagcctcggtctcaaaagggtagttgctgtctagagaggtctggtggagcctgcaaaagtccagctttcaaaggaacacagaagtatgtgtatggaatattagaagatgttgcttttactcttaagttggttcctaggaaaaatagttaaatactgtgactttaaaatgtgagagggttttcaagtactcatttttttaaatgtccaaaattcttgtcaatcagtttgaggtcttgtttgtgtagaactgatattacttaaagtttaaccgaggaatgggagtgaggctctctcataacctattcagaactgacttttaacaataataaattaagtttcaaatatttttaaatgaattgagcaatgttgagttggagtcaagatggccgatcagaaccagaacacctgcagcagctggcaggaagcaggtcatgtggcaaggctatttggggaagggaaaataaaaccactaggtaaacttgtagctgtggtttgaagaagtggttttgaaacactctgtccagccccaccaaaccgaaagtccaggctgagcaaaacaccacctgggtaatttgcatttctaaaataagttgaggattcagccgaaactggagaggtcctcttttaacttattgagttcaaccttttaattttagcttgagtagttctagtttccccaaacttaagtttatcgacttctaaaatgtatttagaattcattttcaaaattaggttatgtaagaaattgaaggactttagtgtctttaatttctaatatatttagaaaacttcttaaaattactctattattcttccctctgattattggtctccattcaattcttttccaatacccgaagcatttacagtgactttgttcatgatcttttttagttgtttgttttgccttactattaagactttgacattctggtcaaaacggcttcacaaatctttttcaagaccactttctgagtattcattttaggagaaagactttttttttaaatgaatgcaattatctagacttatttcagttgaacatgctggttggtggttgagaggacactcagtcagtcagtgacgtgaagggcttctaagccagtccacatgctctgtgtgaactccctctggccctgcttattgttgaatgggccaaaggtctgagaccaggctgctgctgggtaggcctggactttgggtctcccacccagacctgggaatgtatggttgtggcttctgccacccatccacctggctgctcatggaccagccagcctcggtggctttgaaggaacaattccacacaaagactctggacctctccgaaaccaggcaccgcaaatggtaagccagaggcagccacagctgtggctgctgctcttaaagcttgtaaactgtttctgcttaagagggactgagtcttcagtcattgctttagggggagaaagagacatttgtgtgtcttttgagtaccgttgtctgggtcactcacatttaactttccttgaaaaactagtaaaagaaaaatgttgcctgttaaccaataatcatagagctcatggtactttgaggaaatcttagaaagcgtgtatacaattgtctggaattatttcagttaagtgtattagttgaggtactgatgctgtctctacttcagttatacatgtgggtttgaattttgaatctattctggctcttcttaagcagaaaatttagataaaatggatacctcagtggtttttaatggtgggtttaatatagaaggaatttaaattggaagctaatttagaatcagtaaggagggacccaggctaagaaggcaatcctgggattctggaagaaaagatgtttttagtttttatagaaaacactactacattcttgatctacaactcaatgtggtttaatgaatttgaagttgccagtaaatgtacttcctggttgttaaagaatggtatcaaaggacagtgcttagatccgaggtgagtgtgagaggacaggggctggggtatggatacgcagaaggaaggccacagctgtacagaattgagaaagaatagagacctgcagttgaggccagcaggtcggctggactaactctccagccacagtaatgacccagacagagaaagccagactcataaagcttgctgagcaaaattaagggaacaaggttgagagccctagtaagcgaggctctaaaaagcacagctgagctgagatgggtgggcttctctgagtgcttctaaaatgcgctaaactgaggtgattactctgaggtaagcaaagctgggcttgagccaaaatgaagtagactgtaatgaactggaatgagctgggccgctaagctaaactaggctggcttaaccgagatgagccaaactggaatgaacttcattaatctaggttgaatagagctaaactctactgcctacactggactgttctgagctgagatgagctggggtgagctcagctatgctacgctgtgttggggtgagctgatctgaaatgagatactctggagtagctgagatggggtgagatggggtgagctgagctgggctgagctagactgagctgagctagggtgagctgagctgggtgagctgagctaagctggggtgagctgagctgagcttggctgagctagggtgagctgggctgagctggggtgagctgagctgagctggggtaagctgggatgagctggggtgagctgagctgagctggagtgagctgagctgggctgagctggggtgagctgggctgagctgggctgagctgggctgagctggggtgagctgagctggggtgagctgagctgagctggggtgagctgagctgagctggggtgagctggggtgagctgagctggggtgagctgagctgagctggggtgagctgagctggggtgagctgagctgagctggggtgagctgagctgagctgagctgagctgagctggggtgagctgagctgagctgagctggggtgagctggggtgagctgagctgagctggagtgagctgagctgggctgagctggggtgagctgggctgagctggggtgagctgagctgagctgagctgagctggggtgagctgagctgagctggggtgagctgagctggggtgagctgggctgagctgagctgagctgagctgagctgagctgagctgagctgagctgagctgagctgagctgagctgagctgagctgagctgagctggggtgagctgagctgagctgggctgagctggggtgagctgggctgagctgggctgagctgggctgagctggggtgagctgagctggggtgagctgagctgagctgggctgagctgagctgagctggggtgagctgagctgagctggggtgagctgagctgagctgagctggggtgagctgagctgggctgagcagggctgagctggggtgagctgagctgagctggggtgagctgggctgagctgggctgagctgagctgagctgggctgagctgggctgagctgggctgagctgggctgagctgggctgagctggggtgagctgagctgagctggggtgagctggggtgagctgagctggggtgagctgagctggggtgagctgagctgagctggggtgagctgagctggggtgagctgagctgagctggggtgagctgagctgagctggggtgagctgagctagggtgaactgggctgggtgagctggagtgagctgagctgaggtgaactggggtgagccgggatgttttgagttgagctggggtaagatgagctgaactggggtaaactgggatgagctgtggtgagcggagctggattgaactgagctgtgtgagctgagctggggtcagctgagcaagagtgagtagagctggctggccagaaccagaatcaattaggctaagtgagccagattgtgctgggatcagctgtactcagatgagctgggatgaggtaggctgggatgagctgggctagctgacatggattatgtgaggctgagctagcatgggctggcctagctgatgagctaagcttgaatgagcggggctgagctggactcagatgtgctagactgagctgtactggatgatctggtgtagggtgatctggactcaactgggctggctgatgggatgcgccaggttgaactaggctcagataagttaggctgagtagggcctggttgagatggttcgggatgagctgggaaaagatggactcggaccatgaactgggctgagctgggttgggagaccatgaattgagctgaactgagtgcagctgggataaactgggttgagctaagaatagactacctgaattgtgccaaactcggctgggatcaattggaaattatcaggatttagatgagccggactaaactatgctgagctggactggttggatgtgttgaactggcctgctgctgggctggcatagctgagttgaacttaaatgaggaaggctgagcaaggctagcctgcttgcatagagctgaactttagcctagcctgagctggaccagcctgagctgagtaggtctaaactgagttaaaaatcaacagggataatttaacagctaatttaacaagcctgaggtctgagattgaatgagcagagctgggatgaactgaatgagtttcaccaggcctggaccagttaggctaggacctcgttctatagaggcagactgtgtgctacagtggagtttcaagatgattccatgagtcctccccgcccccaacataacccaccttcctcctaccctacacgcctgtctggtgtgtaaatcccagctttgtgtgctgatacagaagcctgagcccctcccccacctccacctacctattactttgggatgagaatagttctcccagccagtgtctcagagggaagccaagcaggacaggcccaaggctacttgagaagccaggatctaggcctctccctgagaacgggtgttcatgcccctagagttggctgaagggccagatccacctactctagaggcatctctccctgtctgtgaaggcttccaaagtcacgttcctgtggctagaaggcagctccatagccctgctgcagtttcgtcctgtataccaggttcacctactaccatatctagccctgcctgccttaagagtagcaacaaggaaatagcagggtgtagagggatctcctgtctgacaggaggcaagaagacagattcttacccctccatttctcttttatccctctctggtcctcagctgtcgcagagatgaaccccaatgtaaatgtgttcgtcccaccacgggatggcttctctggccctgcaccacgcaagtctaaactcatctgcgaggccacgaacttcactccaaaaccgatcacagtatcctggctaaaggatgggaagctcgtggaatctggcttcaccacagatccggtgaccatcgagaacaaaggatccacaccccaaacctacaaggtcataagcacacttaccatctctgaaatcgactggctgaacctgaatgtgtacacctgccgtgtggatcacaggggtctcaccttcttgaagaacgtgtcctccacatgtgctgccagtgagtggcctgggctaagcccaatgcctagccctcccagattagggaagtcctcctacaattatggccaatgccacccagacatggtcatttgctccttgaactttggctccccagagtggccaaggacaagaatgagcaataggcagtagaggggtgagaatcagctggaaggaccagcatcttcccttaagtaggtttgggggatggagactaagcttttttccaacttcacaactagatatgtcataacctgacacagtgttctcttgactgcaggtccctccacagacatcctaaccttcaccatccccccctcctttgccgacatcttcctcagcaagtccgctaacctgacctgtctggtctcaaacctggcaacctatgaaaccctgaatatctcctgggcttctcaaagtggtgaaccactggaaaccaaaattaaaatcatggaaagccatcccaatggcaccttcagtgctaagggtgtggctagtgtttgtgtggaagactggaataacaggaaggaatttgtgtgtactgtgactcacagggatctgccttcaccacagaagaaattcatctcaaaacccaatggtaggtatccccccttcccttcccctccaattgcaggacccttcctgtacctcatagggagggcaggtcctcttccaccctatcctcactactgtcttcatttacagaggtgcacaaacatccacctgctgtgtacctgctgccaccagctcgtgagcaactgaacctgagggagtcagccacagtcacctgcctggtgaagggcttctctcctgcagacatcagtgtgcagtggcttcagagagggcaactcttgccccaagagaagtatgtgaccagtgccccgatgccagagcctggggccccaggcttctactttacccacagcatcctgactgtgacagaggaggaatggaactccggagagacctatacctgtgttgtaggccacgaggccctgccacacctggtgaccgagaggaccgtggacaagtccactggtaaacccacactgtacaatgtctccctgatcatgtctgacacaggcggcacctgctattgaccatgctagcgctcaaccaggcaggccctgggtgtccagttgctctgtgtatgcaaactaaccatgtcagagtgagatgttgcattttataaaaattagaaataaaaaaaatccattcaaacgtcactggttttgattatacaatgctcatgcctgctgagacagttgtgttttgcttgctctgcacacaccctgcatacttgcctccaccctggcccttcctctaccttgccagtttcctccttgtgtgtgaactcagtcaggcttacaacagacagagtatgaacatgcgattcctccagctacttctagatatatggctgaaagcttgcctaacctggtgcaggcagcattcaggcacatatatagacacacatgcatttatacatagatatataggtacacatgtgtagacacatacatgaatgtgtattcatggacacacagacaaaggtacacatatatacacatgagttcatgcgcacacacatgcatggacacttacaaacgccttcagagacaaataggcatagacacacaaccactcacagaaacagataccaatatgcatggtcctgtgtacacagaaacagactataggcaaatatacacaaataaactatatagatacaaagatatgcatatacacacatgtacagaaacatcttcacatgtgtacactaacatgtgaacaggtatagcacacagatacacctggactctgaccagggctgtaatctccaaggctcacggctcagagagcctacactaggctgggtcactgatactcctcaggagcccactctatgattgggagagataaccccaggtacaaagtatgcctatctgtctcaacaccatggggcagaagatactccactaaccacccatgacagaaagttagccttggctgtgtctccattaatagaacacctcagaagaccaatgtgaaattgcctaacccactcacacccaccctgatctccagttcaaaatgcagaaaacataatgcagttgtccaaaagatgccccaaccacacacacacacacacacacacacacacacacacacacacacacacacacacacacacaccatcaaggagcctctgtaaggagtcaccacccaataacactgcctctttgggctcatatcctggacattcttcatattcatatccatttggggcctaggctttagatatccccaagggctcatctttacagggatcagagatcccaataaatgccctggtcccacagcctccctcaggtatctgtctgtttatctcttggtaccaagacccaacattgctggcaggggtaggacaagcaacgcacgggaactctgatcaaagaaagtcatgagatgcctgagtccttcaggaagtaaggagggacaacctctggtatccctgttcttattgctaaagcccaagagacagggagacctgctctaaattctcagtctaaacagcaccgatggcaccacctgctcagggaaagtccagagcacaccaatatcattttgccacagttcctgagtctgcctttacccaggtccatacattgcatctgtcttgcttgctctgctgccccagggctcctggaacaaaggctccaaattagtgtgtcctacagcttggcctgttctgtgcctccgtctagcttgagctattaggggaccagtcaatactcgctaagattctccagaaccatcagggcaccccaacccttatgcaaatgctcagtcaccccaagacttggcttgaccctccctctctgtgtcccttcatagagggggaggtgaatgctgaggaggaaggctttgagaacctgtggaccactgcctccaccttcatcgtcctcttcctcctgagcctcttctacagcaccaccgtcaccctgttcaaggtagtgtggttgtggggctgaggacacagggctgggacagggagtcaccagtcctcactgcctctacctctactccctacaagtggacagcaattcacactgtctctgtcacctgcaggtgaaatgactctcagcatggaaggacagcagagaccaagagatcctcccacagggacactacctctgggcctgggatacctgactgtatgactagtaaacttattcttacgtctttcctgtgttgccctccagcttttatctctgagatggtcttctttctagactgaccaaagactttttgtcaacttgtacaatctgaagcaatgtctggcccacagacagctgagctgtaaacaaatgtcacatggaaataaatactttatcttgtgaactcactttattgtgaaggaatttgttttgtttttcaaacctttcctgcggtgttgacagcccaaggattatctgaatagagcttaggaactggaaatggaacagtgcagtctgatggtacttaagggagaaagagggaaaggaggtgtggaagaagaaaaaagagaagcagagggggaggggagaagggagagggagagggagagggagagggagagggagagggagagggagagagagagagagagagagagagagagagagagagagagagagcatgcactataacttcgtataatgtatgctatacgaagttatctaccgggtaggggaggcgcttttcccaaggcagtctggagcatgcgctttagcagccccgctgggcacttggcgctacacaagtggcctctggctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcagcacgtgttgacaattaatcatcggcatagtatatcggcatagtataatacgacaaggtgaggaactaaaccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagcgggactctggggttcgaataaagaccgaccaagcgacgtctgagagctccctggcgaattcggtaccaataaaagagctttattttcatgatctgtgtgttggtttttgtgtgcggcgcgccgtttaaacgcggataacttcgtataatgtatgctatacgaagttatatgacatcttgttgtggaatagcaaacagagaagactgtttggacatcgatttaaggcaatagaaagtctttactagccaacctgtgactacactgggtgttcaggatctcagtgtagccctgaacctttctcagggtgagattttaggtaaaaattatgttctgggttgacatagttcagttaacaagaacacttagccagaaacagaactacaagacccaaaaagcaaagttaaaacatttacaaactttcctagaactgtatgaactttaatggattaggcgatcgccagcctccagaatggtgggaaataagtttctgttgtttctcagccaccacgtctgtagtatgtggaagtcatcagaatcaaaattgagtcacctgtggttttttttttttctaaatccctgacaaatagagcctaggaaggccaagaagagaagagggttctcatccataaacacttgataacaaaaactatcaccaaggactctacaaaaactgcaactggcacaaagaccatcacaaccttacacagaaagtacttctgtgaggacatcttcccagcaacgggctgtccaacctcagactggcattgcctttgttattggtccttgtagagagggtaattatctcaaagcaatcatgtaatcctcctcatttttcctttgaaagccttggtctccctttgcctccctgaatacgcacatagctgatcatggcaggtgtatcccactgcagtgctctacctccaaatagatatcgcggccgc

SEQ ID NO: 3 - DNA, 1280 bp, Artificial Sequence
  The sequence is synthetized.
  misc_feature (1)..(131): Homologous arm (homologous recombination for linking to 5′- terminal of mouse Ig gamma1) (mouse sequence).
  misc_feature (132)..(176): Human Ig gamma1 Hinge (human sequence).
  misc_feature (295)..(625): Human Ig gamma1 CH2 (human sequence).
  misc_feature (722)..(1044): Human Ig gamma1 CH3 (human sequence).
  misc_feature (1045)..(1280): Homologous arm (homologous recombination for linking to mouse Ig gamma1 3′- terminal) (mouse sequence)
aaatgttggatatcacctactccatgtagagagtcggggacatgggaagggtgcaaaagagcggccttctagaaggtttggtcctgtcctgtcctgtctgacagtgtaatcacatatactttttcttgtagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaatgatcccagtgtccttggagccctctggtcctacaggactctgacacctacctccacccctccctgtgtaaataaagcacccagcactgccttgggaccctgcaataatgtcctggtgatttctgagatgtagagtctagctaggtcatggaatgaggggtctccatggtttgaggcctgagttgtgactaaggaaaaacccataggcctacactgccacacccagcacttttgaattt

SEQ ID NO: 4 - DNA, 24 bp, Artificial Sequence
  The sequence is synthetized.
gacacactttgctacacactcctg

SEQ ID NO: 5 - DNA, 24 bp, Artificial Sequence
  The sequence is synthetized.
gcacagtaatatgtggctgtgtcc

SEQ ID NO: 6 - DNA, 24 bp, Artificial Sequence
  The sequence is synthetized.
tgtttgcaggtgtccagtgtcagg

SEQ ID NO: 7 - DNA, 21 bp, Artificial Sequence
  The sequence is synthetized.
gacaggaagccagccctcagc

SEQ ID NO: 8 - DNA, 24 bp, Artificial Sequence
  The sequence is synthetized.
gggaaagaatgagcaaatgcaagc

SEQ ID NO: 9 - DNA, 25 bp, Artificial Sequence
  The sequence is synthetized.
ttctgtgttcctttgaaagctggac

(19) United States
(12) Patent Application Publication
(10) Pub. No.: US 2021/0017253 A1
(43) Pub. Date: Jan. 21, 2021

(54) NUCLEIC ACID MOLECULES AND APPLICATIONS THEREOF IN PREPARING HUMAN SINGLE-DOMAIN ANTIBODY

(71) Applicant: CHONGQING JINMAIBO BIOTEC CO., LTD, Chongqing (CN)

(72) Inventors: Liangpeng GE, Chongqing (CN); Zuohua LIU, Chongqing (CN); Meng WU, Chongqing (CN); Xiangang ZOU, Chongqing (CN); Xueqin LIU, Chongqing (CN); Xiaoyan YOU, Chongqing (CN); Yuchun DING, Chongqing (CN); Songquan YANG, Chongqing (CN)

(73) Assignee: CHONGQING JINMAIBO BIOTEC CO., LTD, Chongqing (CN)

(21) Appl. No.: 16/982,602

(22) PCT Filed: Apr. 16, 2018
(86) PCT No.: PCT/CN2018/083162

§ 371 Date: Sep. 21, 2020

(30) Foreign Application Priority Data
Mar. 27, 2018 (CN) 201810261005.8

(51) Int. Cl. C07K 16/00; C12N 15/85; A01K 67/027

(52) U.S. Cl. CPC C07K 16/00; C12N 15/8509; A01K 67/0275; C07K 2317/569; C07K 2317/522; A01K 2217/052; C07K 2317/24; C07K 2317/92; C12N 2015/8518; A01K 2267/01; A01K 2227/105; C07K 2317/21

(57) ABSTRACT

Nucleic acid molecules and single-domain antibody applications are provided. The nucleic acid molecules include a transgenic host animal immunoglobulin genes or parts of immunoglobulin genes. Main characters are: it comprises the transgenic host animal IgH 5′-enhancer, IgM switch region (Sμ), and IgM sequences, specifically, all the IgM sequences are originated from the transgenic host animal, and the CH1 sequences of the IgM is deleted (IgM-dCH1). The regulatory control sequences of IgG are also derived from the transgenic host animal including Sγ, TM1, TM2, PolyA sequences, etc., and IgG Cγ sequences (Hinge, CH2 and CH3) are human sequence (Igγ-dCH1). The present invention ensures normal B-cell development in transgenic animal, and the transgenic animal expresses human single-domain antibodies, reducing the subsequent antibody humanization process and improving the druggability of the antibodies.

### CROSS REFERENCE TO THE RELATED APPLICATIONS

This application is the national phase entry of International Application No. PCT/CN2018/083162, filed on Apr. 16, 2018, which is based upon and claims priority to Chinese Patent Application No. 201810261005.8, filed on Mar. 27, 2018, the entire contents of which are incorporated herein by reference.

### TECHNICAL FIELD

The invention belongs to the field of biotechnology, and more specifically, relates to nucleic acid molecules and their applications thereof.

### BACKGROUND

Single-domain antibody, also known as nanobody, or the heavy chain-only antibody. In compar to a normal antibody (150-160 kDa), the single-domain antibody has a much smaller molecular weight of about 50 kDa.

Single-domain antibodies also exist in nature (camels, sharks etc.). The first single-domain antibody was isolated from camel with special usage of camelid heavy-chain variable domain (VHH) sequences. Another method for preparing single-domain antibodies is to generate from a transgenic animal. In 2003 and 2005, Nguyen et al. and Zou et al. respectively constructed transgenic vectors with Igγ2a- CH1 mutation, and the resulting transgenic mice highly expressed single-domain antibodies in vivo. In 2007, Zou et al., reported that in the absence of all mouse light chains (Kappa and Lambda chains), mice could naturally produce single-domain antibodies in vivo.

One of the main characters of single domain antibodies is its small size in molecular weight, which can penetrate and bind to some difficult-access epitopes, which are particularly useful for targets that are inaccessible by the normal antibody, for example, the single-domain antibodies can be used in some cancer diagnostic imaging and treatments. Single-domain antibodies can also be administered orally to treat diarrhea caused by Escherichia coli and gastrointestinal diseases such as inflammatory bowel disease and colorectal cancer. Single-domain antibodies are able to pass through the blood-brain barrier and infiltrate into solid tumors more easily than whole antibodies, and therefore, have the potential to be therapeutic medicine and bispecific antibody treatments to treat brain tumors. Moreover, single-domain antibodies can be easily engineered in vitro with reduced cost during drug development.

### SUMMARY

The objectives of the invention are to provide nucleic acid molecules that can be used to prepare human single-domain antibodies, thereby to reduce the subsequent antibody humanization process and to improve the druggability of the antibodies.

The objectives of the invention are achieved by the following technical steps.

Nucleic acid molecules include immunoglobulin genes or parts of immunoglobulin genes thereof, characterized in that, the nucleic acid molecules include IgG gene (Igγ) and IgG switch region (Sγ), with or without IgM gene (IgHCμ) and IgM switch region (Sμ); wherein both IgM/IgG genes lacking CH1 functions.

Further, the Sμ, Sγ and IgHCμ sequences are derived from the transgenic host animal.

The above-mentioned IgHCμ includes CH2 exon, CH3 exon, CH4 exon, the intron sequences between the CH2 exon and the CH3 exon, and between the CH3 exon and the CH4 exon of the IgM gene, and further includes TM1, TM2, and PolyA signal sequence of the transgenic host animal. Specifically, the structure of the IgHCμ is shown in FIG. 3-1.

The above-mentioned nucleic acid molecule further includes the IgH 5′-enhancer (5′-En) of the transgenic host animal. The enhancer is located at the 5′—the switch region (Sμ or Sγ). Specifically, the structure of the enhancer is shown in FIG. 1 and FIG. 3. The IgH 5′-enhancer is critical for the recombination and transcription of IgH variables, diversities and joins (VDJ) regions, the enhancer can greatly increase the expression of antibodies, mutations and the switch oftransgene. The CH1 sequence of the IgM gene may be deleted, or mutated. In the expression of the IgM gene unit, only exon sequences such as the CH2, CH3, CH4, TM1 and TM2 sequences are used.

In the nucleic acid molecule, the IgM CH2, CH3, CH4, TM1 and TM2 sequences (wherein the CH1 sequence is deleted, IgM-dCH1), and IgH 5′-enhancer and IgM expression regulatory elements are all derived from the transgenic host animal (as shown as light black color in FIG. 3). So the IgH 5′-enhancer (5′-En), the IgM switch region (Sμ) sequence, the IgM CH2, CH3, and CH4 exons (the CH1 exon and its subsequent intron sequence are removed, IgM-dCH1), the TM1, the TM2, the PolyA, and all the sequences therebetween are derived from the transgenic host animal to ensure high expression of the IgM gene and the B-cell development in the transgenic animal. If the host animal is mouse, mouse IgH 5′-enhancer (5′-En), mouse IgM switch region (Sμ) sequences, mouse IgM CH2, CH3, and CH4 exons (the CH1 exon and its subsequent intron sequence are removed, IgM-dCH1), TM1, TM2, PolyA, and all the sequences therebetween are constructed to support the B-cell development and the antibody maturations in mouse.

More specifically, the sequences of the mouse IgH 5′-enhancer, mouse switch region (Sμ) of the IgHCμ, and mouse IgM-dCH1 is listed in SEQ ID NO.2.

The above-mentioned Igγ includes human Hinge exon, CH2 exon, CH3 exon, and sequences therebetween. The IgG gene with the deletion of the CH1 sequences or mutation(s) in the CH1 sequences is designated as Igγ-dCH1. The expression regulatory sequences of Igγ-dCH1 are all derived from the transgenic host animal, including switch region (Sγ) sequence, polyadenylation signal (PolyA), TM1, TM2 and other sequences of the transgenic host animal. If the transgenic animal is mouse, the IgG expression regulatory sequences are from mouse Igγ3, Igγ1, Igγ2a and Igγ2b. Specifically, the structure of the Igγ expression unit is shown in FIG. 1-1. Human Hinge exon, CH2 exon and CH3 exon expression are under the regulatory of the mouse Sγ, TM1, TM2, PolyA, etc., this chimeric expression unit can increase IgG expression level in transgenic host animal and antibody specificity. In detail, the above-mentioned mouse 5′-enhancer, Igγ regulatory sequences and Igγ1-dCH1 sequences are listed SEQ ID NO.1. The human Igγ1 Hinge exon, CH2 exon and CH3 exon sequences with mouse regulatory sequences are listed in SEQ ID NO.3.

The above-mentioned Igγ sequences may include the subtypes of the human Igγ, such as Igγ3, Igγ1, Igγ2 and/or Igγ4; each of the Igγ3, the Igγ1, the Igγ2, and/or the Igγ4 includes Hinge exon, CH2 exon, CH3 exon, etc. (CH1 exon and its subsequent intron sequences are removed, Igγ-dCH1). It is very critical that the regulatory sequences and exons are in prefect order and the exons are in expression reading frame to form single-domain antibody constant region.

If the transgenic host animal is mouse, the mouse Igγ expression regulatory sequences are from mouse Igγ3, Igγ1, Igγ2a, and/or Igγ2b.

The above-mentioned nucleic acid molecules also include IgH 3′-local control region (LCR). The 3′-LCR is derived from the transgenic host animal, such as shown in FIGS. 2, 5, 6 and 7.

The above-mentioned nucleic acid molecules include V-regions of the human IgH heavy chain, D-regions of the human IgH genes; and J-regions of the human IgH gene. The heavy chain V-regions, D-regions and J-regions are all derived from humans, as shown in FIG. 6 and FIG. 7.

For example, specifically, the above-mentioned nucleic acid molecules contain V-regions or modified V-regions of human IgH heavy chain, D-regions or modified D-region of human IgH genes; and J-regions or modified J-regions of human IgH gene, then they are linked to 5′-enhancer (5′-En) of mouse immunoglobulin (IgH) gene, next linked with mouse Igγ switch region (Sγ) sequences, followed by Hinge, CH2 and CH3 sequences of human Igγ, and then mouse PolyA, TM1 and TM2 sequences, finally linked with mouse 3′-locus control region (LCR) of mouse heavy chain IgH (as shown in FIG. 6). The transgenic mice express human single-domain IgG antibodies.

Or, specifically, the above-mentioned nucleic acid molecules contain V-regions or modified V-regions of human IgH heavy chain, D-regions or modified D-region of human IgH genes; and J-regions or modified J-regions of human IgH gene, then they are linked to 5′-enhancer (5′-En) of mouse immunoglobulin (IgH) gene, mouse IgM switch region (Sμ) sequences, and mouse IgM CH2, CH3, CH4 and PolyA, TM1 and TM2 of IgM sequences, next linked with mouse Igγ switch region (Sγ) sequences, followed by Hinge, CH2 and CH3 sequences of human Igγ, mouse PolyA, TM1 and TM2 sequences, finally linked with mouse 3′-locus control region (LCR) of mouse heavy chain IgH (as shown in FIG. 7). The transgenic mice express mouse single-domain IgM and human single-domain IgG antibodies.

The above-mentioned human immunoglobulin genes include part or all of the V-regions, D-regions, and J-regions of the human immunoglobulin (IgH) heavy chain, and further may include human Cμ-region sequences with the deletion or mutations in the CH1 sequence. The above-mentioned mouse immunoglobulin gene includes mouse IgH 5′-enhancer sequences, IgM switch region (Sμ and Sγ) sequences, Polyadenylation signal (PolyA), TM1, TM2, IgH 3′-LCR, etc. of mouse heavy chain.

A vector containing the above-mentioned nucleic acid molecules.

A prokaryote contains the above-mentioned nucleic acid molecules; A cell containing the above-mentioned nucleic acid molecules or vectors, which includes any transgenic cell containing nucleic acid molecules, and further includes but not limited to the lymphocytes, hybridoma cells, antibody-expressing cells, and other cells derived from the transgenic animals.

Human single-domain antibodies generated from the DNA rearrangement and mutations of the above-mentioned nucleic acid molecules. The human single-domain antibodies include any single-domain human antibody derived from the above-mentioned nucleic acid molecules or transgenic animals with the above-mentioned nucleic acid molecules. The invention includes but not limited to proteins, DNAs, mRNA, cDNAs, and any antibody (modified or engineered) derived from the nuclei acid molecules and transgenic animal.

Transgenic animal containing the above-mentioned nucleic acid molecules, vector, cells or antibodies. The animal may be pig, cow, horse, mouse, rat, rabbit, chicken, sheep or other mammals.

The invention contains any application of the above-mentioned DNAs, cDNAs and mRNAs, amino acid sequences, proteins, vectors, hybridoma cells, cell lines and transgenic animals.

In particular, the invention provides transgenic animal obtained by transferring the above-mentioned nuclei acid molecule into animal genome, or the offspring from the cross between the genetically modified animal with another animal with its endogenous immunoglobulin heavy and light chains inactivated, the final transgenic animal can express human single-domain IgG antibodies. Up immunization, the transgenic animal can produce antigen-specific human single-domain IgG antibodies.

For example, includes any single-domain IgM and single-domain human IgG antibodies derived from the above-mentioned nucleic acid molecules, vectors, cells or transgenic animals.

The method for making the transgenic animal with the above-mentioned nucleic acid molecules or vectors includes the following steps:

(1) Obtaining the above-mentioned nucleic acid molecules;

(2) Constructing the nucleic acid molecule vectors;

(3) Introducing the vectors into cells (including ES cells, stem cells, induced pluripotent stem cells and somatic cells) or embryos of a transgenic host animal;

(4) Chimeric production or somatic cell cloning with the cells containing the vectors to generate embryos and then transgenic animal

(5) Breeding to produce heterozygous and homozygous transgenic animals (including mating with a host animal lacking of endogenous immunoglobulin gene functions).

The above-mentioned host animal with a transgenic vector containing the above-mentioned nucleic acid molecules may be pig, cow, horse, mouse, rat, rabbit, chicken, sheep and other mammals. The above-mentioned vectors include yeast artificial chromosome (YAC), bacterial artificial chromosome (BAC), plasmids, DNA fragment, and others. The method for introducing the above-mentioned vectors into cells or embryos includes electroporation, virus infection, liposome-mediation, microinjection, and others.

For some specific embodiments, the above-mentioned nucleic acid molecules listed in SEQ ID NO.1, SEQ ID NO.2 and SEQ ID NO.3 are not intended to limit the scope of the invention. Some non-essential improvements and adjustments to the nucleotide sequences can be made by those skilled in the art, such as deletion, addition, and replacement of some nucleotide sequences.

In the invention, IgM without CH1 function, called IgM-dCH1, can be created by conventional molecular biology methods by those skilled in the art, such as to delete CH1 exon and the intron between CH1 and CH2 exons or to generate mutation(s) in CH1 exon.

### Advantages

1. In the invention, the single-domain antibody transgenic animal is created with modified IgH transgene vector, which the human immunoglobulin heavy chain C-region CH1 sequences are mutated or deleted.

2. In the invention, the human single-domain antibodies are produced in this transgenic animal. Under immunizations of antigens, the transgenic animal can produce high-affinity human single-domain antibodies.

3. In the invention, human single-domain antibodies can be directly generated in vivo, which reduces the subsequent humanization process and improves druggability of the antibodies.

4. The benefits of the invention include: the transgenic vector used to create the transgenic animal contains transgenic host animal IgH 5′-enhancer, the switch region Sμ and the IgM-dCH1 sequences to ensure the normal B-cell development in transgenic animal. The transgenic host animal Igγ switch region (Sγ) sequences, the Igγ polyadenylation signal (PolyA), TM1 sequences, and TM2 sequences are used to regulate the expression of human Igγ-dCH1 (only including the human Igγ Hinge, CH2, CH3, etc.), which is favorable to the DNA recombination and mutation, and B-cell receptor (BCR) signal transduction to support B-cell maturation under the antigen stimulation. In the IgH transgenic animals, human V-regions, D-regions, J-regions and Igγ sequences are used, so the transgenic animals express human single-domain IgG antibodies. Those antibodies do not require subsequent humanization process, and with high the druggability.

5. The main applications of the invention include: Generation of human therapeutic single-domain antibodies, specifically: (1) to treat human diseases, such as brain diseases, tumors, etc.; (2) to construct bispecific antibodies; and (3) to create of chimeric antigen receptor T-cell therapy (CAR-T).

### BRIEF DESCRIPTION OF THE DRAWINGS

FIG. 1: Schematic diagram of the structure and construction of Igγ: Specifically, mouse IgH 5′-enhancer, mouse/human Igγ chimeric expression unit (mouse Sγ, TM1, TM2, PolyA, etc. and human Igγ exons Hinge, CH3, CH4 sequences) is integrated a BAC with human IgG 5′-enhancer, IgM and IgD by homologous recombineering and counter-selection recombineering. Puromycin (Puro) is the selection gene in bacteria and mammal transfections, Lox is a specific 34 base pair sequences.

FIG.2: Schematic diagram of the structure and construction of IgG C-region: Link the BAC above to mouse IgH 3′- Locus Control Region (LCR) by homologous recombineering. the puromycin (Puro) and zeocin (Zeo) selection genes are for bacteria and mammalian cell transfections separately; Frt and Lox are a specific 34 base pair sequence, which the Flpo or CRE expression plasmid or protein can remove its DNA sequences between, resulting only one Lox (34 bp) or Frt (34 bp) sequences reminded in the transgene.

FIG. 3: Schematic diagram of construction of mouse IgH 5′-enhancer and mouse IgM-dCH1: Includes mouse IgH 5′-enhancer, mouse switch region and IgM CH2, CH3, CH4, TM1, TM2, PolyA, etc., and they are integrated to a BAC with human IgG 5′-enhancer, IgM and IgD by homologous recombineering and counter-selection recombineering. Puromycin (Puro) is the selection gene in bacteria and mammal transfections, Lox is a specific 34 base pair sequences.

FIG. 4: Mouse and human Igγ chimeric expression unit: human Igγ Hinge, CH3, CH4 exon sequences is used to replace mouse Igγ CH1, Hinge. CH2 and CH3 sequences (wherein, the human DNA sequences are showed in dark black color, mouse DNA sequences are in light black color).

FIG. 5: Schematic diagram of construction a nuclei acid molecule of IgG-dCH1: link construct in FIG. 3 and FIG. 4 together through homologous recombineering. The puromycin (Puro) and zeocin (Zeo) selection genes are for bacteria and mammalian cell transfections separately; Frt and Lox are specific 34 base pair sequences, which Flpo or CRE expression plasmid or protein can remove its DNA sequences between, resulting only one Lox (34 bp) and Frt (34 bp) sequences reminded in the transgene.

FIG. 6: Main components of a nuclei acid molecule (transgenic animal Ha): Human IgH V-regions, D-regions and J-regions are linked to the above IgG C-region (Igγ-dCH1) and 3′-LCR to form the transgenic DNA construct (wherein, the human DNA sequences are showed in dark black color, mouse DNA sequences are showed in light black color).

FIG. 7: Main components of a nuclei acid molecule (transgenic animal Hb): Human IgH V-regions, D-regions and J-regions are linked to the above IgG C-region (IgM-dCH1 and Igγ-dCH1) and 3′-LCR to form the transgenic DNA construct (wherein, the human DNA sequences are showed in dark black color, mouse DNA sequences are showed in light black color).

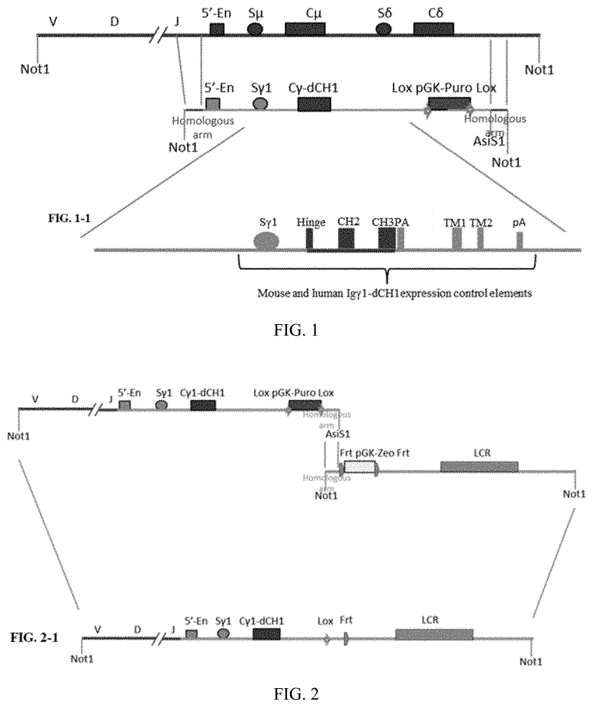

FIG. 1-7: Shows key transgenes in the constructs or vectors.

FIG. 8: Mouse IgH immunoglobulin heavy chain J-region gene target: Wherein the J-regions of the mouse IgH is composed of J1, J2, J3, and J4 genes, and the whole J-region sequence is deleted by the conventional gene targeting; Homogenous mice without the JH sequences cannot produce any mouse-derived Ig (including IgM and IgG).

FIG. 9: The PCR results of human IgHV2-26 with 433 bp PCR product from single-domain antibody transgenic mice (Ha).

FIG. 10: The serum ELISA results from single-domain antibody transgenic mice (Ha).

FIG. 11: The PCR results of human IgHV2-26 with 433 bp PCR product from single-domain antibody transgenic mice (Hb).

FIG. 12: The serum ELISA results from single-domain antibody transgenic mice (Hb).

FIG. 13: Mouse IgH immunoglobulin heavy chain JH gene target
(the size of PCR product of gene targeted mouse is 732 bp, and the size of PCR product of of wild type is 2422 bp).

FIG. 14: The statistical results of antibody specificity and affinity from OVA and HEL immunized transgenic mice.

### DETAILED DESCRIPTION OF THE EMBODIMENTS

The following are specific embodiments for describing the invention in detail. It should be pointed out herein that the following embodiments are only used to further illustrate the invention, and cannot be construed as a limitation to the protection scope of the invention. Some non-essential improvements and adjustments to the invention can be made by those skilled in the art according to the above summary.

### Embodiment

The above nuclei acid molecules containing a modified human immunoglobulin heavy chain are transferred into mouse separately, and then the transgenic mouse is immunized to obtain human single-domain antibodies. The specific steps are as follows.

1. Construction of immunoglobulin gene vectors

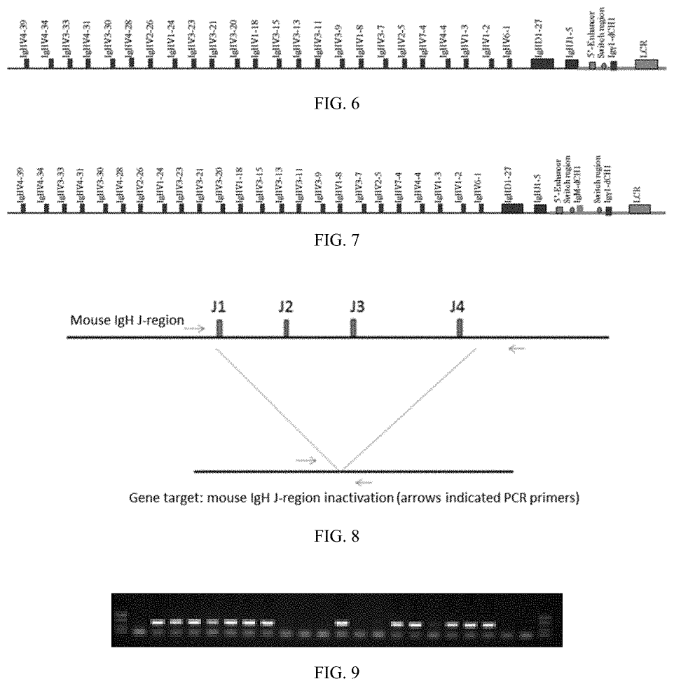

1) Construction of immunoglobulin heavy chain vectors (see FIG. 6)

First, constructing human Igγ expression unit without CH1 sequences: Synthase human Igγ-dCH1 CH1 sequences with homologues arms to ensure that CH2, CH3 and CH4 exons are in reading frame (as FIG. 4); then construct mouse IgH 5′-enhancer and mouse/human Igγ expression unit by counter-selection recombineering; then transfer the above vector into a BAC with human DVJ and IgM, IgD to form a new BAC (as FIG. 1). Then to build mouse IgH 3′-Local Control Region (LCR) with homologous arms, then connect 3′-LCR with another big nuclei acid molecule to form anther BAC (as FIG. 2). Finally, the new C-region is linked to a human heavy chain gene (Ig) in YAC or BAC vector to form the human single-domain heavy chain transgene as shown in FIG. 6 (wherein, the human DNA sequences are showed in dark black color, mouse DNA sequences are in light black color). The human single-domain heavy chain nuclei acid molecule successively includes human immunoglobulin heavy chain V-regions, D-regions and J-regions, mouse IgH 5′-Enhancer, and mouse Sγ, human Igγ-dCH1 and mouse 3′- LCR. Specifically, in the Ig C-region, mouse 5′-enhancer, mouse switch region (Sγ), human Igγ Hinge, CH2, CH3, mouse TM1, TM2 PolyA sequences, etc. to form mouse/human Igγ-dCH1 expression unit. LCR is mouse IgH 3′- local control region.

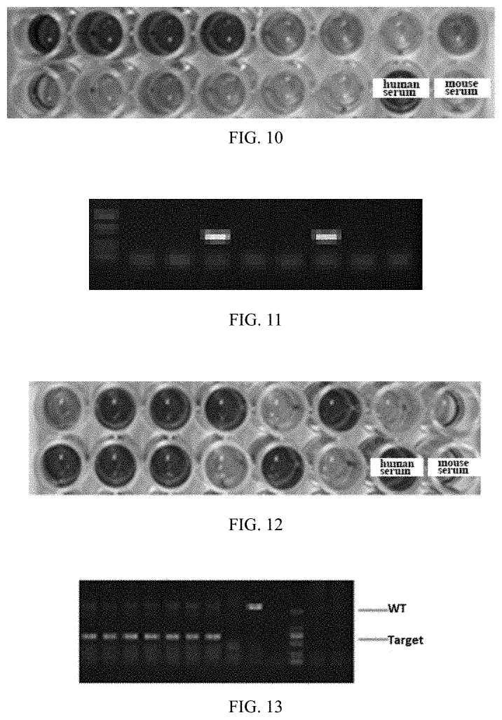

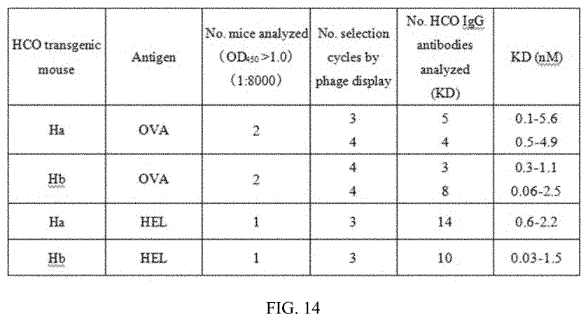

2) Construction of immunoglobulin heavy chain genes (see FIG. 7)

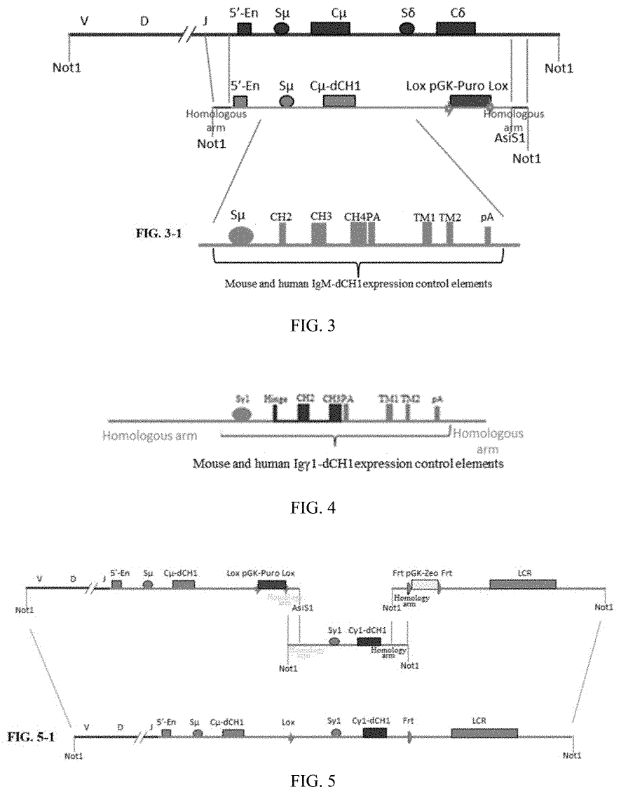

First, constructing mouse IgM expression unit without CH1 sequences: Synthase human IgM-dCH1 sequences with homologues arms to ensure that CH2, CH3 and CH4 exons are in reading frame, then construct mouse IgH 5′-enhancer and mouse/human Igγ expression unit by counter-selection recombineering; then transfer the above vector into a BAC with human DVJ and IgM, IgD to form a new BAC (as FIG. 1); synthase human Igγ-dCH1 sequences with homologues arms to ensure that CH2, CH3 and CH4 exons are in reading frame (FIG. 4); Then to build mouse IgH 3′-Local Control Region (LCR) with homologous arms; link the three vectors together to form a new BAC (as FIG. 5). Finally, the new C-region is linked to a human heavy chain gene (Ig) in YAC or BAC vector to form the human single-domain heavy chain transgene as shown in FIG. 7 (wherein, the human DNA sequences are showed in dark black color, mouse DNA sequences are in light black color). The human single-domain heavy chain nuclei acid molecule successively includes human immunoglobulin heavy chain V-regions, D-regions and J-regions, mouse IgH 5′-Enhancer, mouse Sμ, mouse IgM-dCH1 and mouse Sγ, human Igγ-dCH1 and mouse 3′- LCR (as FIG. 7). Mouse IgM-dCH1 structure is as FIG. 3. IgH 5′-enhancer, switch region (Sμ), IgM CH2, CH3, CH4, TM1, TM2 PolyA sequences, tec. are all originated from mouse; Human Igγ-dCH1 structure is as FIG. 4. Mouse/human Igγ expression unit 9 as FIG. 4) is comprised of mouse switch region (Sγ), human Igγ Hinge, CH2, CH3, mouse TM1, TM2 PolyA sequences. LCR is mouse IgH 3′- local control region.

2. Generations of human single-domain antibody transgenic mice

1) Production of transgenic mice with human immunoglobulin heavy chain genes

A. Generation of transgenic mice with human immunoglobulin heavy chain genes (single-domain antibody transgenic mouse, Ha)

The human immunoglobulin heavy chain vector (as FIG. 6) mentioned in above 1) of method 1 is transferred into mouse genome by conventional transgenic technique. The single-domain antibody transgenic mouse (Ha) with integrated full human immunoglobulin heavy chain vector is confirmed by both PCR and ELISA analysis.

The PCR primers used are as follows.

Human IgH V2-26 PCR:

Primer sequences: SEQ ID NO.4 and SEQ ID NO.5.

The size of the PCR product: 433 bp.

The PCR results are shown in FIG. 9. Note: the genomic DNA PCR results of the human IgHV2-26 transgenic mouse show that the positive mice have a PCR band of 433 bp in size (1% gel electrophoresis).

Human IgHV 3-11 PCR:

Primer sequences: SEQ ID NO.6 and SEQ ID NO.7.

The size of the PCR product is: 686 bp.

ELISA analysis of transgenic mice: The antibodies used for the ELISA detection are: Primary antibody (ab97221, Abcam) for coating and the secondary antibody (AP113P, Millipore) for detection.

ELISA IgG result of transgenic mice as FIG. 10. Notes: human serum and wild type mouse serum are used as controls, see FIG. 9. Note: the human IgG level in transgenic mouse serum is detected by Elisa. The human serum and the wild type mouse serum as controls. The results show that the transgenic mice have high human IgG level in the serum.

B. Generation of transgenic mice with human immunoglobulin heavy chain genes (single-domain antibody transgenic mouse, Hb)

The human immunoglobulin heavy chain vector (as FIG. 7) mentioned in above 2) of method 1 is transferred into mouse genome by conventional transgenic technique. The single-domain antibody transgenic mouse (Hb) with integrated full human immunoglobulin heavy chain vector is confirmed by both PCR and ELISA analysis.

The PCR primers used are as follows.

Human IgH V2-26 PCR:

Primer sequences: SEQ ID NO.4 and SEQ ID NO.5.

The size of the PCR product: 433 bp.

The PCR results are shown in FIG. 11. Note: the genomic DNA PCR results of the human IgHV2-26 transgenic mouse show that the positive mice have a PCR band of 433 bp in size (1% gel electrophoresis).

Human IgHV 3-11 PCR:

Primer sequences: SEQ ID NO.6 and SEQ ID NO.7.

The size of the PCR product is: 686 bp.

ELISA transgenic mice: The antibodies used for the ELISA detection are: Primary antibody (ab97221, Abcam) for coating and the secondary antibody (AP113P, Millipore) for detection.

ELISA IgG result of transgenic mice as FIG. 12. Notes: human serum and wild type mouse serum are used as controls, see FIG. 9. Note: the human IgG level in transgenic mouse serum is detected by Elisa. The human serum and the wild type mouse serum as controls. The results show that the transgenic mice have high human IgG level in the serum.

2) Production of immunoglobulin heavy chain gene knockout mice (see FIG. 8)

Immunoglobulin heavy chain knockout mice are produced by gene targeting. The mouse immunoglobulin heavy chain IgH J-regions is selected as the gene knockout site (see FIG. 8 for the gene knockout location and gene knockout construct) to generate mouse endogenous immunoglobulin heavy chain knockout mouse. Then the knockout mice are screened by both PCR and ELISA analysis.

The primers used for the IgH-JH PCR identification are as follows:

Primer sequences: SEQ ID NO.8 and SEQ ID NO.9.

The PCR results are shown in FIG. 13. PCR products: the size of the PCR product of the JH-region after the gene targeting is 732 bp, while the size of the PCR product of the wild type JH-region is 2422 bp.

ELISA analysis of mouse IgH knock outs: Antibodies, M8644 (Sigma) and A8786 (Sigma) are used, human serum and wild type mouse serum are positive and negative controls.

3) Production of immunoglobulin Kappa light chain knockout mouse (mK⁻⁻ mouse, CN105441455A patent)

4) Breeding to generate human single-domain antibody transgenic mouse

The transgenic mouse Ha) and transgenic mouse Hb) obtained in 2 of the method are crossed with the mice obtained in 2) and 3) of the method respectively. After PCR and ELISA analysis, the resulting single-domain heavy chain transgenic mice express human single-domain IgG, with none (or little) mouse IgG.

ELISA analysis of the serum IgG level of the transgenic mice:

Results: The serum IgG level of wild type mouse is 1-3 mg/mL;

The serum IgG level of human is 3.5-15 mg/mL;

The serum human IgG level of the transgenic mouse (Ha) is 0.001-0.3 mg/mL; and The serum human IgG level of the transgenic mouse (Hb) is 0.01-0.5 mg/mL.

Notes: The human IgG level is low as the Ig-C-region is a modified mouse/human Igγ expression unit, CH1 and its intron are removed (IgG-dCH1), and more, the transgenic mice are kept in a clean and IVC caged environment.

3. EXAMPLES, Generation of single-domain human antibodies

The single-domain human antibody transgenic mice are immunized to produce specific B-cells, in cooperation with phage display technique to generate single-domain antibodies.
A. Example: OVA immunization and single-domain human antibody production
8-week-old single-domain human single-domain antibody transgenic mice are selected for the immunization with OVA.

Primary Immunization:

(1a) OVA (Sigma A7641) antigen is diluted with PBS to a final concentration of 2 mg/mL, then 20 μg of CpG (ODN1826, tlrl-1826, Invivogen) is added, and then an appropriate amount of aluminum hydroxide (vac-alu-50, Invivogen) is added to allow a concentration of the aluminum hydroxide to be 1%.

(2a) 0.75 mL of the antigen prepared in step (1a) is mixed with a complete Freund's adjuvant (CFA, Sigma F5881) in a ratio of 1:1, and emulsified with a MIXPAC™ syringe. Each mouse is immunized by subcutaneous injection at a dose of 200 μL each (0.2 mg).

Secondary Immunization:

(1b) On the 21^{st} day after the primary immunization, a secondary immunization is performed. The antigen is diluted with PBS to a final concentration of 1.0 mg/mL, then 10 μg of CpG is added, and an appropriate amount of aluminum hydroxide is added to allow a concentration of the aluminum hydroxide to be 1%.

(2b) 0.75 mL of the antigen prepared in step (1b) is mixed with an incomplete Freund's adjuvant (IFA) in a ratio of 1:1, and emulsified with a MIXPAC™ syringe. Each mouse is immunized by intraperitoneal injection at a dose of 200 μL (0.1 mg).

Third Immunization:

(1c) On the 21^{st} day after the secondary immunization, a 3^{rd} immunization is performed. The antigen is diluted with PBS to a final concentration of 1.0 mg/mL, then 10 μg of CpG is added, and an appropriate amount of aluminum hydroxide is added to allow a concentration of the aluminum hydroxide to be 1%.

(2c) The antigen protein prepared according to the method in step (1c) is injected directly. Each mouse is immunized by intraperitoneal injection at a dose of 200 μL (0.1 mg).

Fourth Immunization:

(1d) On the 21^{st} day after the 3^{rd} immunization, a 4^{th} immunization is performed. The antigen is diluted with PBS to a final concentration of 1.0 mg/mL, then 10 μg of CpG is added, and an appropriate amount of aluminum hydroxide is added to allow a concentration of the aluminum hydroxide to be 1%.

(2d) The antigen protein prepared according to the method in step (1d) is injected directly. Each mouse is immunized by intraperitoneal injection at a dose of 200 μL (0.1 mg).

Booster Immunization:

On the 21^{st} day after the 4^{th} immunization, mice with satisfactory serum ELISA human IgG titer are given a booster immunization, and then splenic B-cells are obtained for hybridoma fusion, culture and screening.

1) Mouse serum enzyme-linked immunosorbent assay (ELISA)

On the 10^{th} day after the 4^{th} immunization, the blood of the mice is taken for ELISA analysis of mouse IgG and human IgG titer of the immunized mouse serum.

Mouse serum IgG analysis: 96-well plates are embedded with an antigen OVA, and the specific anti-human IgG-HRP antibody (Millipore, AP113P) is used.

Transgenic mouse serum titer with OC450 more than 1.0 (at 1:8000 dilution) after immunization is selected for B-cell collection and antibody generation.

2) Generation of single-domain antibodies.

Mouse splenic B-cells are prepared and high antigen-specific expressed B-cells (hIgG⁺CD138⁺Ag⁺, FACS sorting etc.) are isolated and collected for mRNA preparation, cDNA transcription and phage display to mine the single-domain antibodies with high specificity and affinity (KD=0.03-5.6 nM, as shown in Table 14).

What is claimed is:

1. Nucleic acid molecules comprising immunoglobulin genes or parts of the immunoglobulin genes, wherein, the nucleic acid molecules comprises an IgG gene (Igγ), an IgG switch region (Sγ) and an IgM gene (IgHCμ) and an IgM switch region (Sμ), wherein, the IgM gene and the IgG gene do not have a CH1 function.

2. The nucleic acid molecules according to claim 1, wherein the Sμ, the Sγ and the IgHCμ are derived from a transgenic host animal.

3. The nucleic acid molecules according to claim 1, wherein the IgHCμ comprises an IgM CH2 exon, an IgM CH3 exon, an IgM CH4 exon, intron sequences between the IgM CH2 exon and the IgM CH3 exon, and between the IgM CH3 exon and the IgM CH4 exon, a TM1, a TM2 and PolyA signal sequences of the transgenic host animal.

4. (canceled)

5. The nucleic acid molecules according to claim 3, wherein the Igγ comprises a Hinge exon, a CH2 exon, a CH3 exon and intron sequences between the Hinge exon and the CH2 exon, and between the CH2 exon and the CH3 exon a TM1, a TM2 and PolyA signal sequences.

6. The nucleic acid molecules according to claim 3, wherein nucleotide sequence the TM1, the TM2 and the PolyA signal sequences are from the transgenic host animal.

7. (canceled)

8. The nucleic acid molecules according to claim 1, wherein IgH heavy chain 5′-enhancer is from a transgenic host animal.

9. The nucleic acid molecules according to claim 1, wherein Sγ comprises Sγ1, Sγ3, Sγ2a and/or Sγ2b.

10. The nucleic acid molecules according to claim 1, wherein the Sμ sequences are listed in SEQ ID No.2. (2550) . . . (4451).

11. The nucleic acid molecules according to claim 1, wherein 5′-enhancer sequences are listed in SEQ ID No.2. (433) . . . (1444).

12. The nucleic acid molecules according to claims 1, wherein the IgG gene (Igγ) is comprised of a transgenic host animal IgG/human IgG chimeric expression unit or human IgG sequences.

13. The nucleic acid molecules according to claim 12, wherein the Igγ comprises the human Igγ subtype or the human Igγ subtypes, and the transgenic host animal Igγ subtype or the transgenic host animal Igγ subtypes.

14. The nucleic acid molecules according to claim 13, wherein the human Igγ subtypes comprises Igγ3, Igγ1, Igγ2 and Igγ4.

15. The nucleic acid molecules according to claims 1, wherein the nucleic acid molecules comprise a transgenic host animal or human IgH heavy chain 3′- local control region.

16. The nucleic acid molecules according to claims 1, wherein the nucleic acid molecules comprise all or parts of V-regions of human IgH heavy chain, all or parts of D-regions of human IgH, and all or parts of J-regions of human IgH.

17. (canceled)

18. A vector, containing the nucleic acid molecules according to claims 1.

19. A cell, comprising the nucleic acid molecules according to claim 1 or a vector, wherein the vector contains the nucleic acid molecules.

20. A human antibody derived from the nucleic acid molecules according to claim 1, a vector, or a cell, wherein the vector contains the nucleic acid molecules, and the cell comprises the nucleic acid molecules or the vector.

21. Methods of using the nucleic acid molecule of any one of claims 1-17, the vector of claim 18, or the cell of claim 19 in encoding DNA, cDNA, mRNA, expressing amino acid sequences, proteins, vectors, cultivating hybridoma cells, cell lines and transgenic animals, and preparing humanized single-domain antibodies.

22. A method for preparing a transgenic animal with the nucleic acid molecules according to claim 1, a vector, or a cell, wherein the vector contains the nucleic acid molecules, the cell comprises the nucleic acid molecules or the vector, and the method comprises the following steps:
(1) obtaining the nucleic acid molecules;
(2) constructing the nucleic acid molecules into an untreated vector to obtain the vector;
(3) introducing the vector into transgenic cells or embryos of the host animal;
(4) embryos and transgenic animals generated from chimeric production or somatic cell cloning with the cells containing the vector.
(5) breeding to producing heterozygous and homozygous transgenic animals.

23. The nucleic acid molecules according to claim 1, further comprised of IgH heavy chain 5′-enhancer of a transgenic host animal.
